(19) United States
(12) Patent Application Publication
(10) Pub. No.: US 2025/0345066 A1
(43) Pub. Date: Nov. 13, 2025

(54) TRANSCATHETER PULMONARY FLOW RESTRICTOR

(71) Applicants: Elaheh Malakan Rad, Tehran (IR); Ziyad Hijazi, San Diego, CA (US)

(72) Inventors: Elaheh Malakan Rad, Tehran (IR); Ziyad Hijazi, San Diego, CA (US)

(21) Appl. No.: 19/197,046

(22) Filed: May 2, 2025

Related U.S. Application Data
(60) Provisional application No. 63/646,035, filed on May 13, 2024.

(51) Int. Cl. A61B 17/12; A61B 17/00; A61L 33/00; A61L 33/08

(52) U.S. Cl. CPC A61B 17/12172; A61B 17/12036; A61B 17/12109; A61B 17/12177; A61L 33/0011; A61L 33/08; A61B 2017/00889; A61B 2017/00893; A61B 2017/12095

(57) ABSTRACT

A transcatheter pulmonary flow restrictor device comprising a disk-shaped body, which is concave on the proximal side and convex on the distal side, made from tightly interwoven ultra-thin mesh wires with shape memory and no substantial gaps to restrict pulmonary blood flow. The disk-shaped body has two symmetrically positioned openings on opposite sides of the disk center for passage of blood flow. The device may further comprise a central screw on the proximal side, an appendage on the distal side opposite the central screw, a heparin-based bioactive coating over the mesh wires, an antibacterial layer within or over the mesh wires, and two radio-opaque markers associated with the openings for proper positioning of the device. Materials of the device vary based on the intended duration of use in the pulmonary arterial branches, including the use of smart, bioresponsive, and biodegradable materials with an inverse relationship between pulmonary arterial pressure and device degradation.

### CROSS-REFERENCE TO RELATED APPLICATIONS

Priority is hereby claimed to provisional application Ser. No. 63/646,035, filed May 13, 2024, which is incorporated herein by reference.

### BACKGROUND

Pulmonary artery banding, first introduced by Muller and Danimann in 1952, is a palliative surgical procedure originally designed to limit pulmonary blood flow in young children with congenital heart disease when complete surgical repair is not feasible. (See Muller and Danimann. “The treatment of certain congenital malformations of the heart by the creation of pulmonic stenosis to reduce pulmonary hypertension and excessive pulmonary blood flow; a preliminary report.” Surg Gynecol Obstet. 1952, 95: 213-219.)

Over the years, the applications and techniques for pulmonary artery banding have evolved significantly. In 2006, transcatheter pulmonary artery banding emerged as a compelling concept when Mollet et al. reported the first instance of a percutaneously implanted pulmonary flow restrictor in sheep (Mollet et al. “Development of a device for transcatheter pulmonary artery banding: evaluation in animals.” Eur Heart J., 2006, 27:3065-3072). The device consisted of a conduit-shaped PolyTetraFluoroEthylene (PTFE) membrane-covered self-expanding stent with a central constriction.

Following this, various transcatheter devices have been developed. For example, U.S. Pat. No. 6,638,257, granted to Amplatz, describes a transcatheter pulmonary flow restrictor comprising a tubular structure with a disk containing two holes located in the center of the tubular occluder.

In 2007, Boucek and colleagues in the United States introduced pulmonary flow regulators known as Joeys, which were implanted into the branches of the pulmonary artery in lambs (Boucek et al. “Percutaneous selective pulmonary artery bands (Joeys) in a pulmonary overcirculation model.” Catheter Cardiovasc Interv., 2007, 70: 98-104). These devices were made from 0.018-inch (about 0.46 millimeters) Nitinol wire and measured 6 to 10 millimeters in diameter and 6 to 8 millimeters in length. Each device had a thin Dacron web (composed of polyester materials) embedded in the wire mesh except for the flow lumen. Two design variants were available, differing in the configuration of the internal orifices for flow: one with a single, eccentrically positioned large hole, and another with two smaller, symmetrically arranged holes of similar size. Insertion of the device was achieved through femoral vein access.

Additionally, fenestrated Medtronic MVP™ Micro Vascular Plugs (Medtronic, Inc., Minneapolis, MN) have been utilized as transcatheter pulmonary flow restrictors. These devices were successfully deployed in newborn piglets as part of a preclinical study exploring their utility for both implantation and explantation (Khan et al. “Utility of the Medtronic microvascular plug as a transcatheter implantable and explantable pulmonary artery flow restrictor in a swine model.” Catheter Cardiovasc Interv. 2019, 93: 1320-1328).

However, in humans, only two types of devices have been used so far for percutaneous restriction of pulmonary blood flow: (1) Fenestrated Amplatzer occluder (Gorenflo and Gewillig. “A flow restrictor implanted percutaneously across a loose pulmonary artery band.” Catheter Cardiovasc Interv. 2011, 77: 696-699); and (2) Fenestrated MVP™ Micro Vascular Plug (Schranz et al. “Hypoplastic Left Heart Stage I: No Norwood, No Hybrid.” Circulation. 2020, 142: 1402-1404; Schranz et al. “Functional regeneration of dilated cardiomyopathy by transcatheter bilateral pulmonary artery banding: first-in-human case series.” Eur Heart J Case Rep. 2023, 7: ytad052; Kizilski et al. “Transcatheter Pulmonary Artery Banding in High-Risk Neonates: In-Vitro Study Provoked by Initial Clinical Experience.” Cardiovasc Eng Technol. 2023, 14: 640-654).

Nevertheless, currently available devices exhibit several limitations (Malakan Rad and Hajazi. “Transcatheter Pulmonary Flow Restrictors: Current Trends and Future Perspectives.” Catheter Cardiovasc Interv. 2025, 105: 165-180). These limitations include the need for a large delivery sheath for implantation, limited patient eligibility due to a narrow range of available device sizes, the inability to close the device once deployed, and the lack of adjustability to accommodate pulmonary artery growth over time. Additional concerns include the risks of embolization and proximal migration, as well as the difficulty, or in some cases, impossibility of retrieving the device without damaging the endoluminal pulmonary arterial wall. Device removal is often hindered by endothelialization and adhesion to the arterial wall, frequently necessitating surgical reconstruction of the pulmonary artery. Other shortcomings include thrombus formation, partial device collapse due to peri-device leaks, and the potential for infection.

Accordingly, there remains a need for improved devices and methods for transcatheter pulmonary flow restriction that overcome the limitations of current technologies.

### SUMMARY

Disclosed herein is a transcatheter pulmonary flow restrictor device. The device comprises a disk-shaped body formed from interwoven mesh wires, wherein the disk-shaped body comprises at least one opening extending through a thickness of the disk-shaped body; the interwoven mesh wires are arranged at a strand density sufficient to inhibit fluid flow through the mesh, such that fluid flow through the disk-shaped body occurs solely through the at least one opening; and the disk-shaped body is configured to have a preset expanded configuration and exhibit a shape memory property, such that the disk-shaped body is deformable to a reduced dimension for delivery to a blood vessel, and self-expandable to the preset expanded configuration upon deployment within the blood vessel.

Due to the high strand density, the disk-shaped body is free of polyester fabric that is incorporated into or interwoven with the mesh wires to facilitate occlusion of blood flow.

In preferred versions, the interwoven mesh wires are arranged in precise contact with one another to form a continuous, gap-free mesh.

In preferred versions, the mesh wires are ultra-thin wires having a diameter ranging from about 0.001 inch to about 0.004 inch.

The primary materials of the mesh wire are not limited. In one version, the mesh wires are formed primarily of nitinol. In alternative versions, the mesh wires are formed primarily of a biodegradable material selected from the group consisting of polydioxanone (PDO), poly-L-lactic acid (PLLA), polyglycolic acid (PGA), polylactic acid (PLA), hydrogels, and silk fibroin. In yet another alternative version, the biodegradable material exhibits a degradation rate inversely correlated with pulmonary arterial pressure, wherein the degradation rate is controlled via incorporation of mechanosensitive elements, pressure-sensitive polymers, or coatings responsive to mechanical stress.

Preferably, the disk-shaped body is configured to be concave on a proximal side and convex on a distal side, the concave side facing in the direction of pulmonary arterial blood flow.

In certain versions, the disk-shaped body has a circumferential peripheral border having a triangular cross-sectional profile with curved and filleted transitions to minimize contact with an endoluminal surface of a pulmonary artery.

In preferred versions, the at least one opening comprises two openings positioned symmetrically on opposite sides of the center of the disk-shaped body, each opening having a circular cross-sectional shape. The device may further comprise two radio-opaque markers each associated with one of the two openings and aligned along a central axis of the disk-shaped body.

In preferred versions, the device further comprises a central screw attached to a proximal side of the disk-shaped body to facilitate delivery and retrieval of the device, and an appendage attached to a distal side of the disk-shaped body to facilitate positioning of the device and impeding proximal migration post-deployment.

In certain versions, the appendage is spherical and formed of a material having a greater density than the mesh wires.

In certain versions, the central screw and the appendage are formed primarily of a biodegradable material selected from the group consisting of polydioxanone (PDO), poly-L-lactic acid (PLLA), polyglycolic acid (PGA), polylactic acid (PLA), hydrogels, and silk fibroin. In this specific version, the appendage has a greater density than the central screw to create a distal weight bias, facilitating positioning of the device.

In certain versions, the biodegradable material of the central screw and the appendage exhibits a degradation rate inversely correlated with pulmonary arterial pressure, wherein the degradation rate is controlled via incorporation of mechanosensitive elements, pressure-sensitive polymers, or coatings responsive to mechanical stress. In this specific version, the appendage has a greater density than the central screw to create a distal weight bias, facilitating positioning of the device.

In certain versions, the device further comprises a coating applied over the mesh wires, the coating being configured to reduce inflammation, wherein the coating may comprise a heparin-based bioactive coating.

In certain versions, the device further comprises an antibacterial layer positioned within or over the mesh wires, wherein the antibacterial layer may comprise an elutable antimicrobial material.

The objects and advantages of the disclosure will appear more fully from the following detailed description of the embodiment of the disclosure made in conjunction with the accompanying drawings.

### BRIEF DESCRIPTION OF THE DRAWINGS

FIG. 1 is a top plan view of a transcatheter pulmonary flow restrictor (TPFR) device according to the present disclosure.

FIG. 2 is a bottom plan view of the device shown in FIG. 1.

FIG. 3 is a cross-sectional view of the device, taken along line A-A of FIG. 1, showing a bisected view to reveal its internal structure.

FIG. 4 is a longitudinal cross-sectional view of the device, taken along line B-B of FIG. 1, illustrating internal components and structure along the length of the device.

FIG. 5 is a longitudinal cross-sectional view shown in FIG. 4, illustrating the primary dimensions of the device, including the diameter of the disk-shaped body (a), the thickness of the disk-shaped body (b), the length of the narrowed and filleted edge (c), and the angle of concavity (0).

FIG. 6 displays an enlarged view of the peripheral border of the dish-shaped body, illustrating a triangular cross-sectional profile with curved and filleted transitions.

FIG. 7 shows a schematic illustration of a human heart, showing the main pulmonary artery originating from the right ventricle and branching into the right and left pulmonary arteries.

FIGS. 8 and 9 depict deployment of TPFR devices in a scenario where no major branch arises proximally in either pulmonary artery. The TPFR devices are deployed within the distal segments of both the right and left pulmonary arteries at their respective pre-branching regions. FIG. 9 is an enlarged view of the area shown in FIG. 8.

FIGS. 10 and 11 depict deployment of TPFR devices in a scenario where a major branch arises proximally in the right pulmonary artery. Accordingly, one TPFR is deployed within the distal segment of the right pulmonary artery, a second TPFR is deployed within the proximal segment of the first major branch, and a third TPFR is deployed within the distal segment of the left pulmonary artery at its pre-branching region. FIG. 11 is an enlarged view of the area shown in FIG. 10.

FIGS. 12 and 13 depict an alternative deployment of TPFR devices in the scenario shown in FIGS. 10 and 11. One TPFR is deployed within the proximal segment of the right pulmonary artery before the origin of the first major branch, and another TPFR is deployed distally before bifurcation in the left pulmonary artery. FIG. 13 is an enlarged view of the area shown in FIG. 12.

### DETAILED DESCRIPTION

### Definitions

In the following description, certain terms have been used for brevity, clarity, and understanding. No unnecessary limitations are to be inferred therefrom beyond the requirement of the prior art because such terms are used for descriptive purposes and are intended to be broadly construed. The different configurations and systems described herein may be used alone or in combination with other configurations and systems. It is to be expected that various equivalents, alternatives and modifications are possible within the scope of the foregoing description.

Any version of any component of the disclosure may be used with any other component of the disclosure. The elements described herein can be used in any combination whether explicitly described or not.

As used herein, the singular forms “a,” “an,” and “the” include plural referents unless the content clearly dictates otherwise.

As used herein, the term “or” is an inclusive “or” operator and is equivalent to the term “and/or” unless the context clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

The devices of the present disclosure can comprise, consist of, or consist essentially of the essential elements and limitations described herein, as well as any additional or optional components, or limitations described herein or otherwise useful in the art. The disclosure provided herein suitably may be practiced in the absence of any element which is not specifically disclosed herein.

While this disclosure may be embodied in many forms, what is described in detail herein is a specific preferred embodiment of the disclosure. The present disclosure is an exemplification of the principles of the disclosure and is not intended to limit the disclosure to the particular embodiments illustrated. It is to be understood that this disclosure is not limited to the particular examples, configurations, materials, and arrangements disclosed herein as such configurations, materials, and arrangements may vary somewhat. It is also understood that the terminology used herein is used for the purpose of describing particular embodiments only and is not intended to be limiting since the scope of the present disclosure will be limited to only the appended claims and equivalents thereof.

As used herein, “distal” refers to a direction away from the delivery catheter within the pulmonary artery, while “proximal” refers to a direction closer to the delivery catheter. For purposes of this disclosure, the distal-proximal axis is also aligned with the direction of blood flow through the pulmonary artery, with distal being downstream and proximal being upstream relative to the blood flow.

As used herein, “shape memory” refers to the property of a material to recover and maintain a predetermined configuration after being deformed, including during storage or delivery through a catheter. Upon release from constraint and/or exposure to physiological conditions, the material resumes its intended shape. Materials exhibiting shape memory include, but are not limited to, shape memory alloys such as nickel-titanium (nitinol), biodegradable polymers such as polydioxanone (PDO), polylactic acid (PLA), polyglycolic acid (PGA), copolymers of lactic and glycolic acid (PLGA), hydrogels, and silk fibroins.

### The Transcatheter Pulmonary Flow Restrictor (TPFR)

The present disclosure is directed to a medical device for restricting pulmonary blood flow by partially occluding the pulmonary artery, thereby reducing excessive blood flow to the lungs or other organs. In certain embodiments, the device is used to restrict pulmonary blood flow in small patients with biventricular hearts and complex or Swiss-cheese ventricular septal defects, as well as in children with univentricular hearts and unrestricted pulmonary blood flow. The device is intended for use as an initial palliative procedure to facilitate progression toward the Fontan procedure or to address any clinical condition requiring short-term pulmonary artery banding. Such conditions include elevating the afterload on the morphological left ventricle prior to an arterial switch operation in patients with transposition of the great arteries (TGA) or in L-TGA patients undergoing a staged double switch procedure, reducing tricuspid regurgitation in patients with congenitally corrected TGA and a systemic right ventricle, and enhancing left ventricular function or regeneration in patients with dilated cardiomyopathy and preserved right ventricular systolic function.

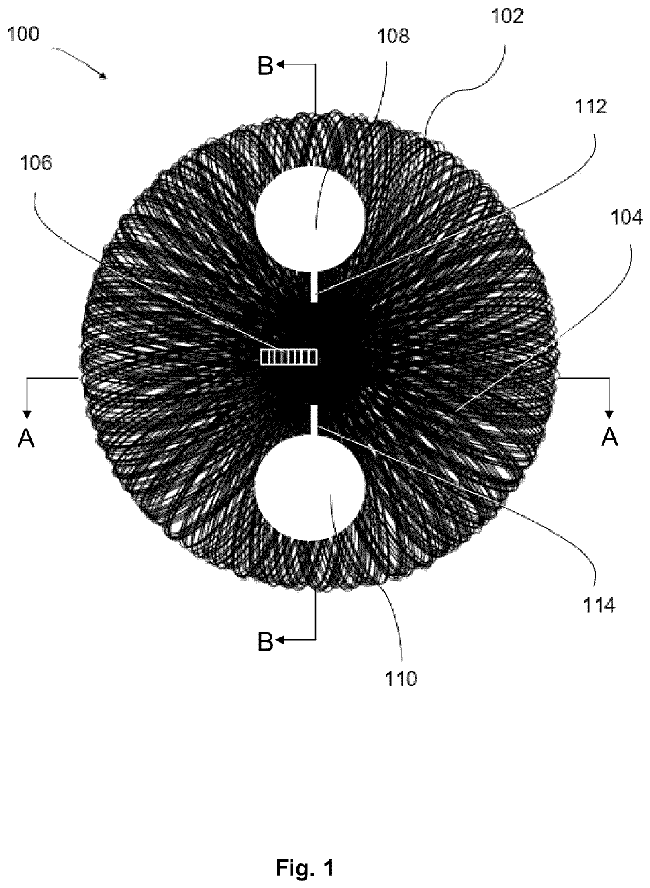

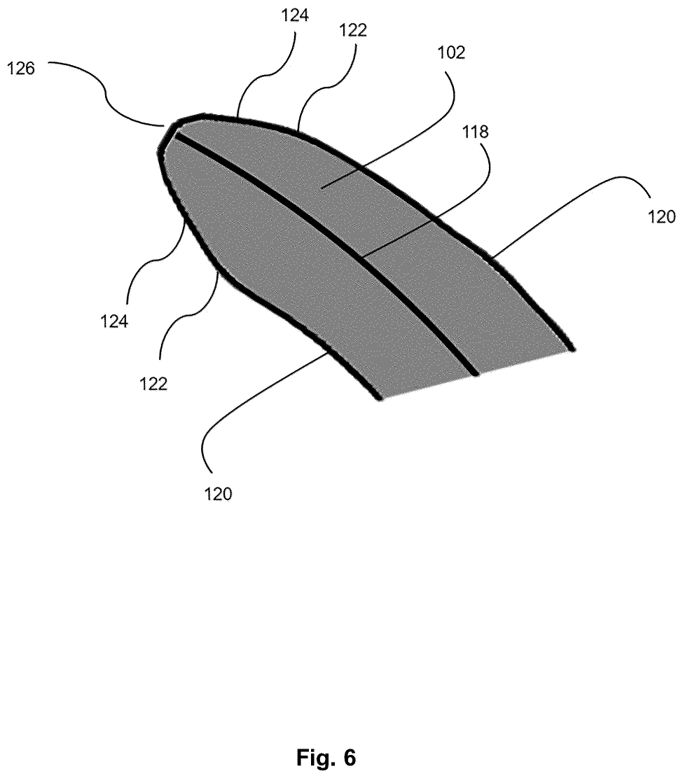

Referring to FIGS. 1-6, a preferred embodiment of the present disclosure includes a transcatheter pulmonary flow restrictor (TPFR) device 100 comprising a single, self-centering and self-expandable disk-shaped body 102 (interchangeably referred to as the “disk”) formed from tightly interwoven mesh wires 104 exhibiting shape memory properties.

The tightly interwoven mesh wires 104 are arranged at a strand density sufficient to inhibit fluid flow through the mesh, such that substantially no macroscopic interstitial gaps exist between adjacent wires, and fluid flow through the disk-shaped body 102 occurs solely through at least one opening, such as openings 108 and 110. Preferably, the wires 104 are arranged in precise contact with one another to form a continuous, gap-free mesh, with no measurable spacing between adjacent strands. Due to the high strand density, no additional fibrous materials, such as polyester fabric, are required within the mesh to facilitate occlusion of blood flow.

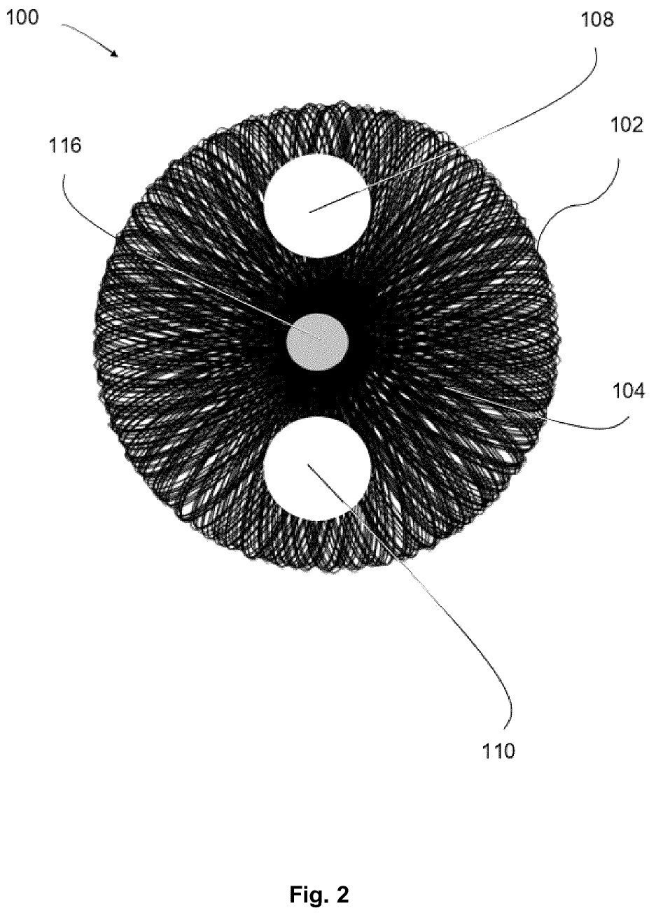

The disk-shaped body 102 is configured to have a preset expanded configuration and exhibits a shape memory property, such that the disk-shaped body is deformable to a reduced dimension for delivery to a blood vessel, and self-expandable to the preset expanded configuration upon deployment within the blood vessel. FIGS. 1-2 show the device 100 in a fully expanded state, in which the disk-shaped body is a disk-like structure with no tubular extension.

Preferably, the tightly woven mesh wires 104 are ultra-thin wires having a diameter ranging from about 0.001 inch to about 0.004 inch (about 0.025 mm to 0.10 mm). To compensate for the reduced wire thickness in terms of mechanical strength, the tightly interwoven and substantially gap-free configuration of the mesh provides sufficient structural integrity and fracture resistance for the device. The ultra-thin mesh wires 104 provide enhanced flexibility and allow for safe accommodation within the pulmonary artery, particularly when the device is deployed at 30% to 50% oversize relative to the vessel diameter to allow for patient growth. The reduced wire diameter facilitates ease of expansion in response to natural enlargement of the pulmonary artery over time. Additionally, the thin wire profile lowers the risk of vascular erosion and minimizes the likelihood of triggering an inflammatory response. It also reduces the potential for the device to embed into the endoluminal surface of the pulmonary artery, thereby facilitating atraumatic removal without causing damage to the vessel wall.

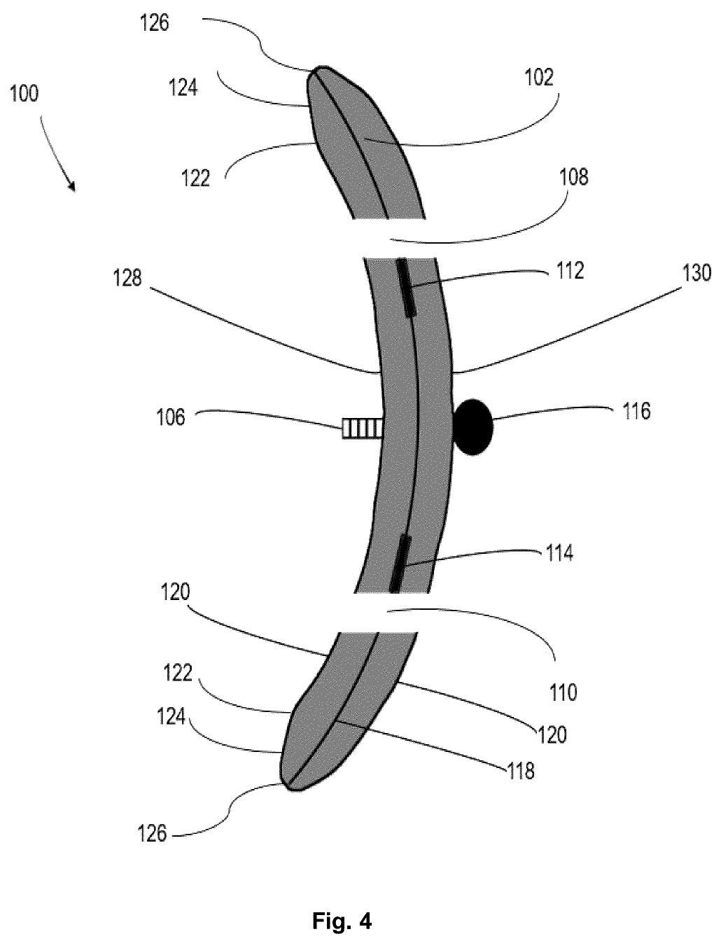

In preferred versions, the disk-shaped body 102 is configured to be concave on the proximal side 128 and convex on the distal side 130 (FIG. 4). The concave surface 128 is oriented proximally, facing the direction of pulmonary arterial blood flow, while the convex surface 130 is positioned distally before the first major pulmonary arterial branch. This non-flat, contoured configuration of the disk-shaped body 102 serves to counteract proximal displacement of the device 100 following deployment and reduces the risk of thrombus formation by presenting a shallow concave angle, which minimizes flow disruption and stasis. This configuration also prevents proximal embolization of the device toward the pulmonary valve.

In preferred versions, the disk-shaped body 102 has a circumferential peripheral border having a generally triangular cross-sectional profile with curved and filleted transitions 122, 124, and 126 (FIGS. 4-6). This peripheral design minimizes the contact area 126 between the device 100 and the endoluminal surface of the pulmonary arterial wall. Minimizing contact in this manner is critical for reducing the risk of inflammation and preventing incorporation of the device into the vessel wall in a way that could complicate retrieval. The design thereby facilitates atraumatic removal of the device, without causing damage to the pulmonary arterial wall.

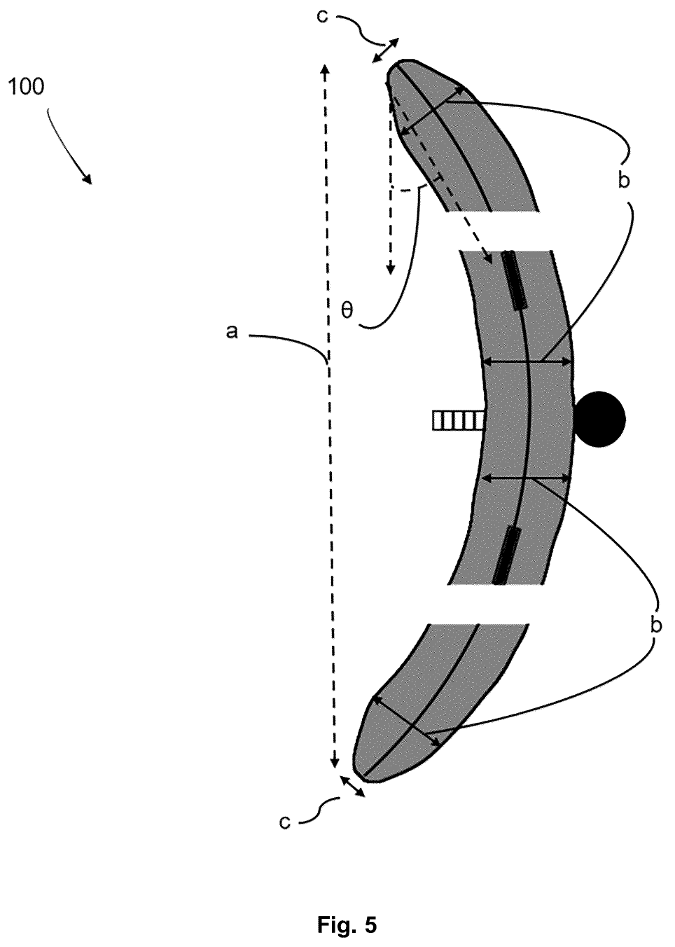

Major dimensional parameters of the disk-shaped body 102 are illustrated in FIG. 5, including the diameter of the disk-shaped body 102 (“a”), the thickness of the disk-shaped body 102 (“b”), the length of the narrowed and filleted edge 126 (“c”), and the angle of concavity (θ). In preferred versions, when fully expanded, the disk-shaped body 102 has a diameter (“a”) ranging from about 4 mm to about 20 mm, and a thickness (“b”) of about 5 mm. However, the thickness of the peripheral section that contacts the endoluminal pulmonary arterial wall (“c”) is about 2 mm. The concavity of the disk-shaped body 102 preferably forms an angle (θ) of about 30° between the central axis of the disk and the line defining the concave surface.

To select an appropriately sized device, the diameter of the target pulmonary arterial branch, measured proximally to the first major branch, is determined and increased by approximately 130% to 150% to ensure effective anchoring and accommodation of patient growth.

As noted above, the disk-shaped body 102 includes at least one opening extending through its thickness to permit blood flow into the pulmonary vascular bed. In preferred versions, the disk-shaped body 102 comprises two symmetrically positioned openings 108 and 110 located on opposite sides of the center of the disk. The openings preferably have identical circular cross-sectional shapes. Preferably, opening 108 is positioned above the center of the disk-shaped body, while opening 110 is positioned below the center, with both openings vertically spaced apart and aligned along the central axis of the disk-shaped body (FIGS. 1-2 and 4). These openings are configured to control and direct blood flow through the device, while the surrounding mesh structure inhibits flow through the remainder of the disk.

In preferred versions, the device 100 further comprises two radio-opaque markers 112 and 114, each associated with one of the openings 108 and 110, respectively. Each marker extends from its corresponding opening and points toward the center of the disk-shaped body 102. Specifically, the marker 112 extends downward from the base of the upper opening 108, while the marker 114 extends upward from the top of the lower opening 110. The markers are aligned along the central vertical axis of the disk, providing clear fluoroscopic visibility to aid in orientation. This alignment facilitates accurate positioning and deployment of the device, ensuring the intended alignment of the openings for effective control of pulmonary blood flow. The radiopaque markers may be made from any suitable materials. Non-limiting examples include platinum or platinum-iridium alloy, which are widely used in cardiovascular implants due to their excellent radiopacity, biocompatibility, and corrosion resistance. The markers are micro-welded or mechanically fixed to the wire mesh of the disk during device manufacturing, ensuring secure integration without compromising the structural or functional integrity of the device.

The device 100 further comprises a central screw 106 positioned on the proximal side of the disk-shaped body 102 (FIGS. 1-2 and 4). The central screw 106 is attached to the disk-shaped body 102 at or near its center, and is configured to facilitate delivery and retrieval of the device. The presence of the central screw 106 enables seamless attachment to a delivery system, and allows for retrieval using a snare, in a manner similar to conventional Amplatzer® occluder devices (St. Jude Medical, Cardiology Division, Inc., St. Paul, MN).

The device 100 further comprises an appendage 116 attached to the distal side of the disk-shaped body 102 at or near its center, and positioned directly opposite the central screw 106 located on the proximal side (FIGS. 1-2 and 4). Preferably, the appendage 116 has a spherical shape. The appendage 116 may be made of stainless steel. The relatively high density of stainless steel, typically ranging from about 7.9 to 8.0 g/cm³, compared to the density of the mesh wires formed from materials such as nitinol (about 6.45 g/cm³), facilitates distal positioning of the device within the pulmonary artery branch and serves to impede proximal migration post-deployment. In certain embodiments, the spherical appendage 116 has a density that is 1.5 times of the central screw 106, and its increased mass provides a stabilizing counterweight on the distal side of the device. This design strategically leverages the difference in material densities to ensure device stability, prevent proximal displacement, and maintain proper orientation within the pulmonary vasculature.

The appendage 116 may be welded to the distal side of the central axis, which is continuous and coaxially aligned with the central screw 106. This configuration ensures structural integrity and precise alignment along the device's longitudinal axis. During device assembly, the wire mesh of the disk is concentrically braided around this central axis, integrating the appendage and the disk into a unified structure that preserves both flexibility and anchoring stability. Importantly, the appendage 116 is deliberately compact to allow smooth accommodation within the delivery sheath during transcatheter implantation.

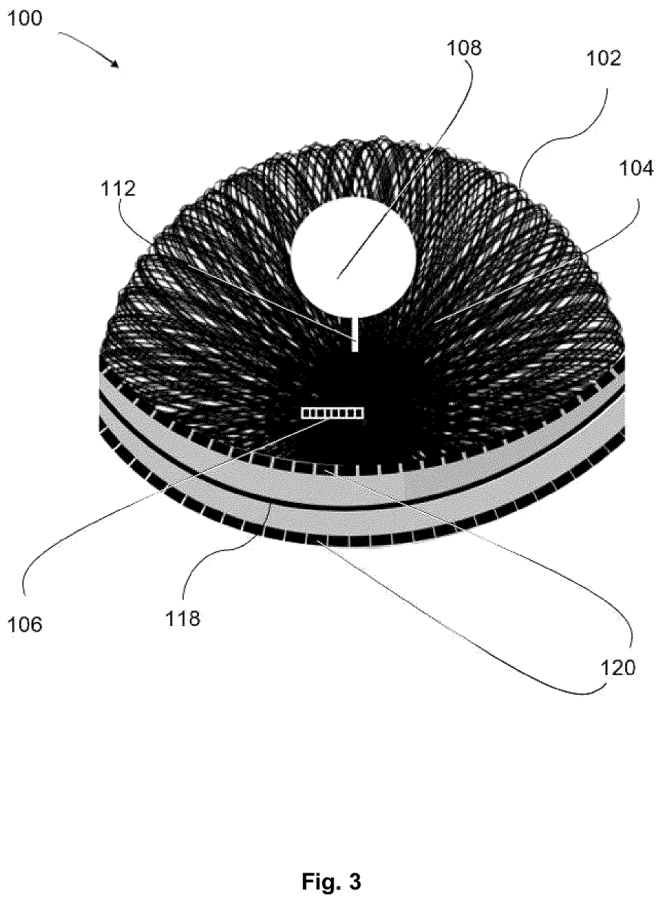

In certain versions, the device 100 comprises a coating applied over the mesh wires, shown as numeral 120 in FIGS. 3-4, which is configured to reduce inflammation. Suitable coatings include, but are not limited to, a bioactive surface covering known as CARMEDA® coating (Carmeda AB, Solna, Sweden), which is a heparin-based coating designed to impede thrombosis and reduce inflammation by inhibiting blood clot formation. By preventing thrombosis, the coating also indirectly reduces the risk of infection associated with implanted devices.

In certain versions, the device 100 comprises a thin layer of antibacterial material within or over the mesh wires, shown as numeral 118 in FIGS. 3-4. Suitable materials include, but are not limited to, TYRX™ adsorbable antibacterial material (Medtronic, Inc., Minneapolis, MN), which is designed to elute antimicrobial agents over time to prevent bacterial colonization. This antibacterial layer reduces the risk of infection by impeding microbial adhesion, and may also contribute to lowering inflammation and thrombosis, thereby enhancing biocompatibility and integration with the pulmonary arterial wall.

The device 100 is self-centering. The self-centering capability is primarily achieved through two key design features. First, the presence of the central screw 106 and the symmetrically positioned appendage 116 on the opposite side of the single-disc structure result in a balanced distribution of mass and mechanical force. This intrinsic symmetry enables the device to naturally align itself with the center of the atrial septal defect upon deployment. Second, the device is composed of a shape-memory alloy, such as Nitinol, which allows it to resume its pre-formed configuration once released from the delivery system. Provided that the device size is appropriately matched to the defect, these features collectively ensure stable, centrally positioned within the pulmonary arterial branch.

The self-centering design of the device ensures that it aligns precisely within the lumen of the right and left pulmonary arterial branches. This alignment is critical to prevent blood from leaking around the edges of the device, thereby ensuring that flow is directed exclusively through the intended openings in the device itself. By fully sealing the arterial lumen except at these controlled points, the device can regulate pulmonary blood flow as intended. Without proper centering, blood may bypass the flow-restricting mechanism, leading to suboptimal pressure or volume control.

The device 100 may be made from any materials suitable for its intended function. According to the anticipated duration of the device staying within the pulmonary arterial branch prior to retrieval, the device 100 may be provided in the following four specific embodiments, each utilizing materials selected for their suitability to the intended use and duration:

Embodiment A is intended for use in the pulmonary artery for three months or less.

Embodiment B is intended for use in the pulmonary artery for over three months and up to 12 months.

Embodiment C is intended for use in the pulmonary artery for over 12 months and up to two years.

Embodiment D, referred to as Intelligent Transcatheter Pulmonary Flow Restrictor (ITPFR), is made from biodegradable biomaterials and is engineered to exhibit an inverse relationship between biodegradability rate and pulmonary arterial pressure. This smart, pressure-responsive design allows the device to adapt dynamically to physiological conditions and is particularly suitable for small children with complex and/or Swiss-cheese ventricular septal defects, offering adaptive functionality in response to physiological conditions.

All four embodiments of TPFR comprise the same structural elements and are available in multiple sizes, including a smaller version designed for deployment in a branch arising from the proximal right pulmonary artery, or less commonly, the left pulmonary artery, to prevent pulmonary overflow in the affected segments.

The embodiments vary primarily in the choice of alloy or biomaterial used for the disk-shaped body 102, depending on the intended duration of placement within the pulmonary arterial branches. In cases where prolonged duration is required and retrieval at a later stage may pose a risk of injury to the endoluminal surface of the pulmonary arterial wall, biodegradable biomaterials are used to enable safe and controlled degradation of the device over time.

Accordingly, different materials are selected for each embodiment. Embodiment A utilizes nitinol mesh wires as the primary material for the disk-shaped body, suitable for short-term placement (≤3 months). Embodiment B utilizes the synthetic biodegradable polymer polydioxanone (PDO), designed for intermediate use (>3 months to ≤12 months). Embodiment C utilizes poly-L-lactic acid (PLLA), another synthetic biodegradable polymer, for extended use (>12 months to ≤2 years). While PDO and PLLA do not match nitinol in shape memory and superelasticity, they exhibit similar properties to a lesser extent, making them suitable for temporary implants requiring controlled degradation.

The main feature of Embodiment D is its fabrication from smart, biodegradable, bioresponsive materials. These include polyglycolic acid (PGA), polylactic acid (PLA), PDO, PLLA, hydrogels, and silk fibroin enhanced for bioresponsiveness. The key feature of Embodiment D is its ability to adjust its degradation rate inversely with pulmonary arterial pressure, allowing dynamic adaptation in response to physiological changes. This pressure-responsive degradability is achieved through one or more of the following methods: incorporating mechanosensitive elements, blending with pressure-sensitive polymers, linking degradation to biochemical markers of mechanical stress, and developing smart coatings. Key implementation considerations include biocompatibility, safety, predictability, and control. Any approach must ensure that the materials used remain biocompatible and do not release harmful substances upon degradation or when subjected to mechanical stress.

Additionally, the central screw 106, the appendage 116, and the radio-opaque markers 112 and 114 may be fabricated from materials selected specifically for each embodiment.

In Embodiment A, the central screw 106 may be made of medical grade 316L stainless steel. The appendage 116 may also be made of stainless steel. The radio-opaque markers 112 and 114 may be made from a platinum-iridium alloy, typically composed of 80% platinum and 20% iridium.

In Embodiments B, C, and D, where the mesh wire is biodegradable, these components may also be made of the same biodegradable material as the mesh wire. In such designs, the appendage 116 creates a distal weight bias, making the distal side of the device heavier than the proximal side. This weight distribution aids in proper deployment and positioning of the device within the pulmonary artery branch. Accordingly, the materials selected for the appendage 116 are preferably biocompatible, predictably biodegradable, have sufficient density to provide mass, and occupy a small volume to fit through the transcatheter delivery sheath. In Embodiment B, the appendage is preferably fabricated from magnesium-based alloys such as WE43 or Mg—Ca, which offer an optimal biodegradable alternative. In embodiments C and D, iron-based biodegradable alloys, particularly iron-manganese (Fe—Mn) systems, are preferably used to fabricate the appendage.

### Use of the TPFR

The present disclosure provides a device for restricting pulmonary blood flow by deploying the device in the right and left pulmonary arteries before the first significant branch.

Deployment and retrieval of the TPFR are similar to those of conventional Amplatzer® occluders (St. Jude Medical, Cardiology Division, St. Paul, MN). The device may be introduced through either the femoral vein or the jugular vein, allowing for flexible access routes depending on patient anatomy and procedural requirements.

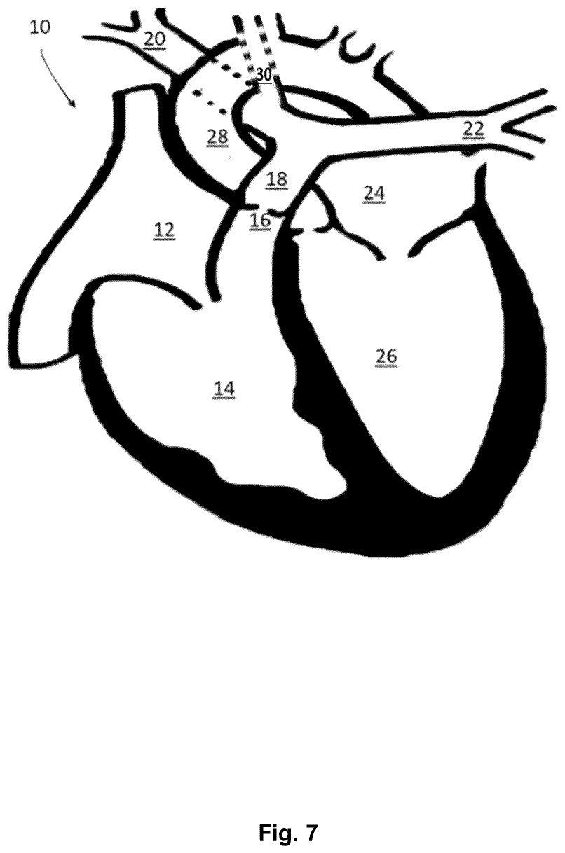

FIG. 7 illustrates a human heart 10, depicting the right atrium 12, right ventricle 14, pulmonary valve 16, main pulmonary artery 18, right pulmonary artery branch 20 and its first significant branch 30, as well as the left pulmonary artery branch 22. These arteries are responsible for delivering blood to the pulmonary vascular bed. The diagram also identifies the left atrium 24, left ventricle 26, and aorta 28.

The present disclosure describes three primary deployment configurations of the TPFR. The deployment strategy depends on the anatomical configuration of the right and left pulmonary arteries 20 and 22, particularly whether a major branch arises proximally within either artery.

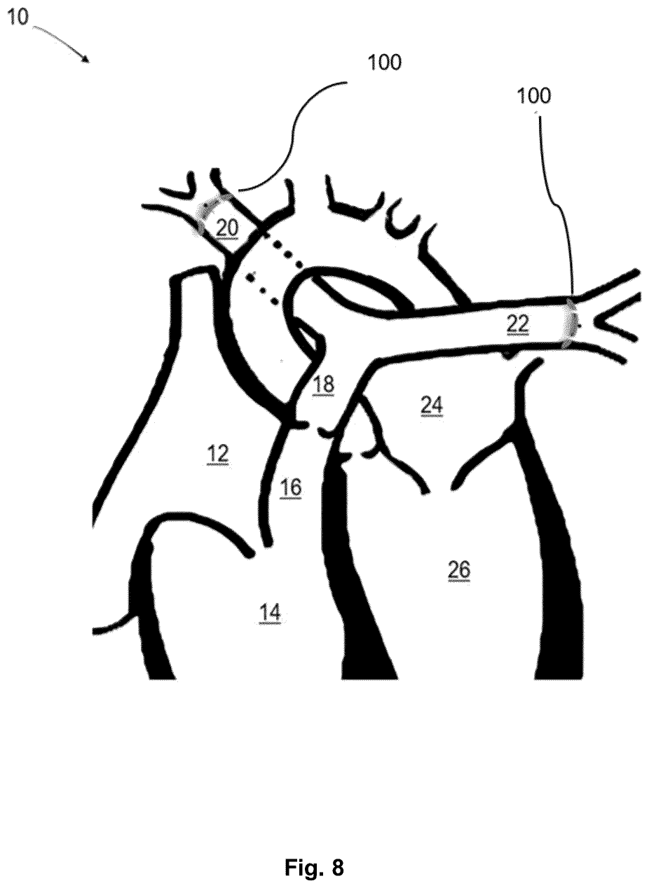

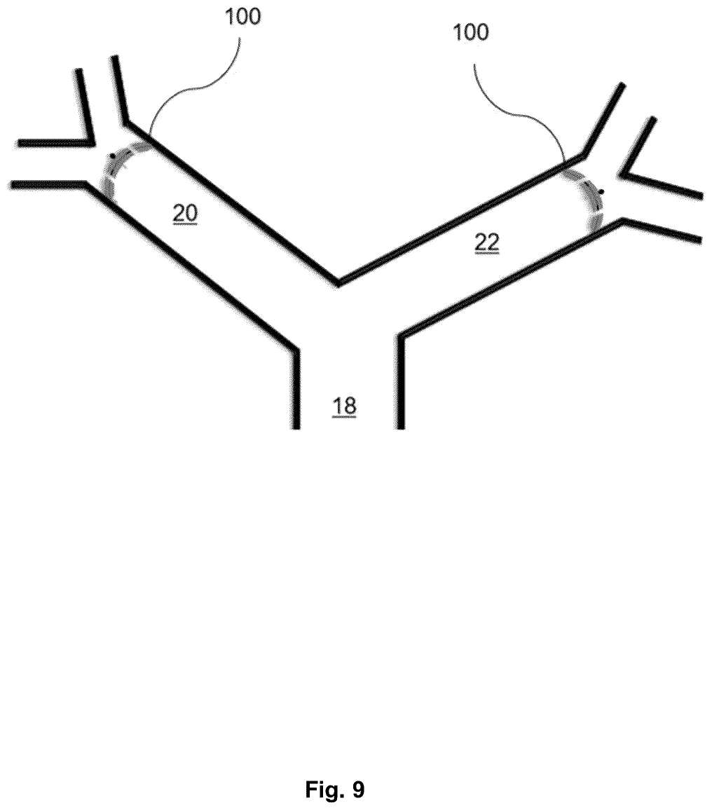

Referring to FIGS. 8 and 9, in the first configuration, no major branch arises proximally in either pulmonary artery. The TPFR is deployed within the distal segments of both the right and left pulmonary arteries (20, 22), at their respective pre-branching regions.

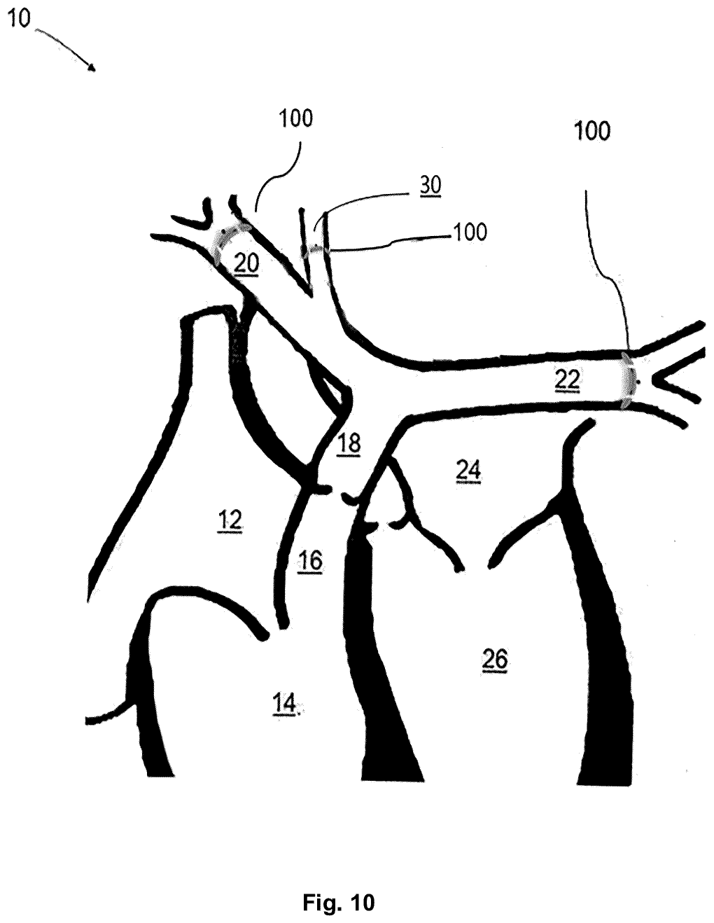

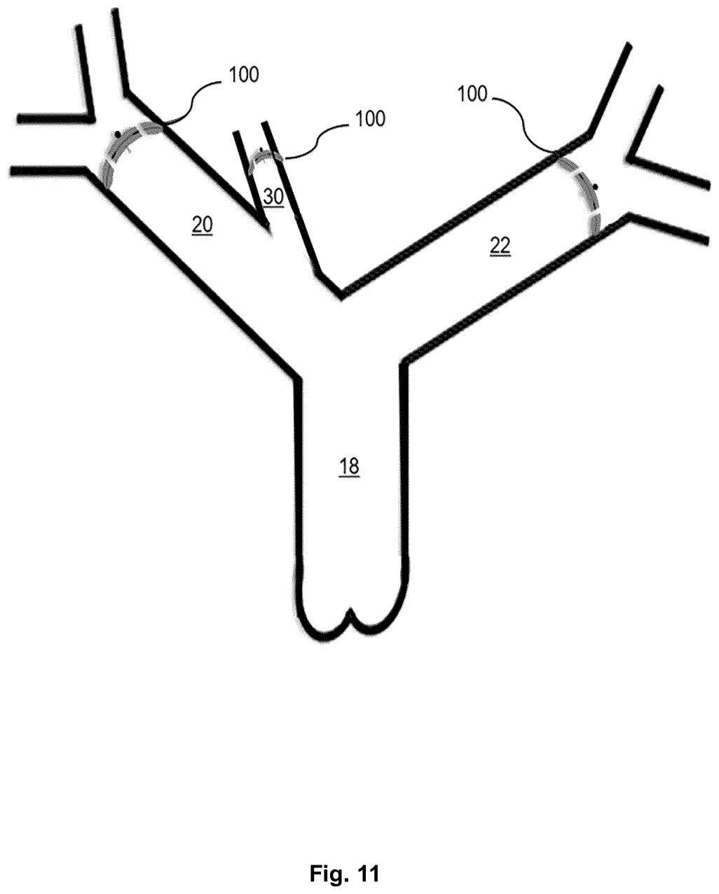

Referring to FIGS. 10 and 11, in the second configuration, a major branch 30 arises proximally in the right pulmonary artery 20. Accordingly, one TPFR is deployed within the distal segment of the right pulmonary artery 20, and a second TPFR is deployed within the proximal segment of the first major branch 30. The same approach can be applied to the left pulmonary artery 22 if it has a proximally arising major branch. If not, a TPFR is deployed within the distal segment of the left pulmonary artery 22 at its pre-branching region, as shown in FIGS. 10 and 11. The sizing of the TPFR for a proximally arising major branch follows the same principle as for the right or left pulmonary artery, with the device diameter being 30% to 50% larger than the internal diameter of the target vessel.

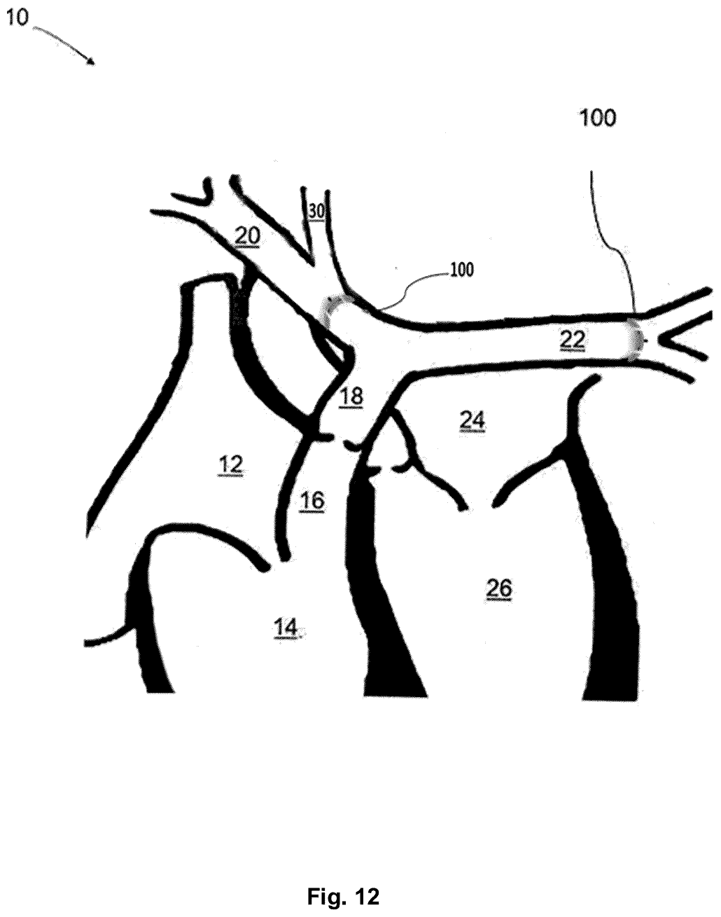

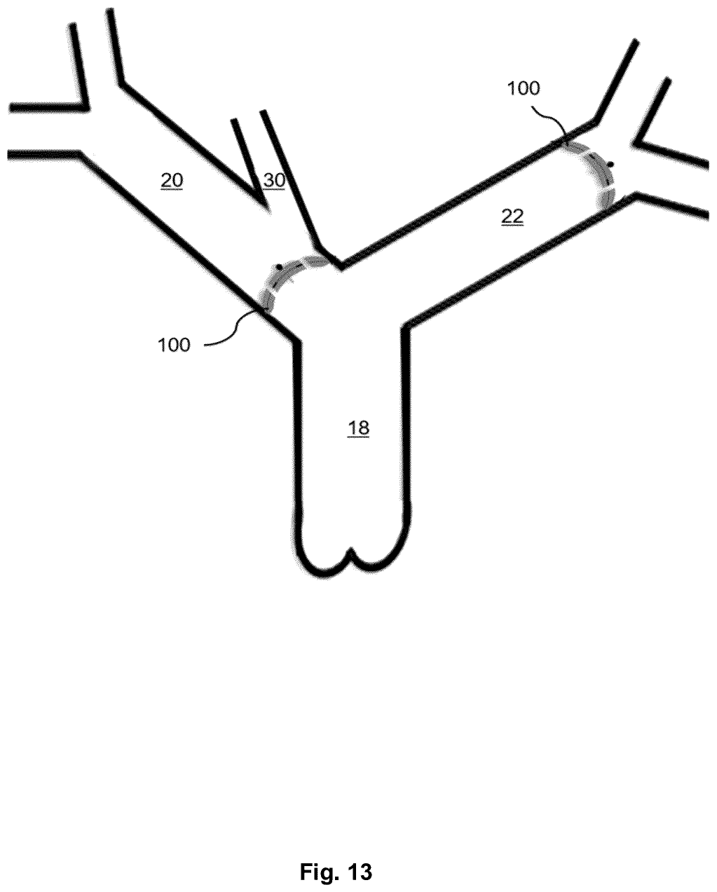

Referring to FIGS. 12 and 13, the third configuration is an alternative to the second configuration when a major branch 30 arises proximally in the right pulmonary artery 20. In this configuration, a TPFR is deployed within the proximal segment of the right pulmonary artery 20, before the origin of the first major branch 30. In contrast, if no major branch arises proximally in the left pulmonary artery 22, the TPFR is deployed distally before bifurcation in the left pulmonary artery.

What is claimed is:

1. A transcatheter pulmonary flow restrictor device, comprising:
a disk-shaped body formed from interwoven mesh wires, wherein:
the disk-shaped body comprises at least one opening extending through a thickness of the disk-shaped body;
the interwoven mesh wires are arranged at a strand density sufficient to inhibit fluid flow through the mesh, such that fluid flow through the disk-shaped body occurs solely through the at least one opening; and
the disk-shaped body is configured to have a preset expanded configuration and exhibit a shape memory property, such that the disk-shaped body is deformable to a reduced dimension for delivery to a blood vessel, and self-expandable to the preset expanded configuration upon deployment within the blood vessel.

2. The device of claim 1, wherein the disk-shaped body is free of polyester fabric that is incorporated into or interwoven with the mesh wires.

3. The device of claim 1, wherein the interwoven mesh wires are arranged in precise contact with one another to form a continuous, gap-free mesh.

4. The device of claim 1, wherein the mesh wires are ultra-thin wires having a diameter ranging from about 0.001 inch to about 0.004 inch.

5. The device of claim 1, wherein the mesh wires are formed primarily of nitinol.

6. The device of claim 1, wherein the mesh wires are formed primarily of a biodegradable material selected from the group consisting of polydioxanone (PDO), poly-L-lactic acid (PLLA), polyglycolic acid (PGA), polylactic acid (PLA), hydrogels, and silk fibroin.

7. The device of claim 6, wherein the biodegradable material exhibits a degradation rate inversely correlated with pulmonary arterial pressure, wherein the degradation rate is controlled via incorporation of mechanosensitive elements, pressure-sensitive polymers, or coatings responsive to mechanical stress.

8. The device of claim 1, wherein the disk-shaped body is configured to be concave on a proximal side and convex on a distal side, the concave side facing in the direction of pulmonary arterial blood flow.

9. The device of claim 1, wherein the disk-shaped body has a circumferential peripheral border having a triangular cross-sectional profile with curved and filleted transitions to minimize contact with an endoluminal surface of a pulmonary artery.

10. The device of claim 1, wherein the at least one opening comprises two openings positioned symmetrically on opposite sides of the center of the disk-shaped body, each opening having a circular cross-sectional shape.

11. The device of claim 10, further comprising two radio-opaque markers each associated with one of the two openings and aligned along a central axis of the disk-shaped body.

12. The device of claim 1, further comprising a central screw attached to a proximal side of the disk-shaped body to facilitate delivery and retrieval of the device, and an appendage attached to a distal side of the disk-shaped body to facilitate positioning of the device and impeding proximal migration post-deployment.

13. The device of claim 12, wherein the appendage is spherical and formed of a material having a greater density than the mesh wires.

14. The device of claim 12, wherein the central screw and the appendage are formed primarily of a biodegradable material selected from the group consisting of polydioxanone (PDO), poly-L-lactic acid (PLLA), polyglycolic acid (PGA), polylactic acid (PLA), hydrogels, and silk fibroin.

15. The device of claim 14, wherein the appendage has a greater density than the central screw to create a distal weight bias, facilitating positioning of the device.

16. The device of claim 14, wherein the biodegradable material exhibits a degradation rate inversely correlated with pulmonary arterial pressure, wherein the degradation rate is controlled via incorporation of mechanosensitive elements, pressure-sensitive polymers, or coatings responsive to mechanical stress.

17. The device of claim 16, wherein the appendage has a greater density than the central screw to create a distal weight bias, facilitating positioning of the device.

18. The device of claim 1, further comprising a coating applied over the mesh wires, the coating being configured to reduce inflammation.

19. The device of claim 18, wherein the coating comprises a heparin-based bioactive coating.

20. The device of claim 1, further comprising an antibacterial layer positioned within or over the mesh wires.

21. The device of claim 20, wherein the antibacterial layer comprises an elutable antimicrobial material.
